(19) United States
(12) Patent Application Publication
(10) Pub. No.: US 2002/0032375 A1
(43) Pub. Date: Mar. 14, 2002

(54) Method and system for visualizing a body volume and computer program product

(75) Inventors: Thomas Bauch, Munchen (DE); Nils Frielinghaus, Geretsried (DE)

Correspondence Address: Don W. Bulson, Esq., Renner, Otto, Boisselle & Sklar, P.L.L., 1621 Euclid Ave., 19th Floor, Cleveland, OH, 44115, US

(73) Assignee: BrainLAB AG

(21) Appl. No.: 09/859,697

(22) Filed: May 17, 2001

(30) Foreign Application Priority Data
Sep. 11, 2000 (DE) 100 44 801.1

(51) Int. Cl. A61B 5/00

(52) U.S. Cl. 600/407; 128/920

(57) ABSTRACT

The invention relates to a method for two- or three-dimensionally visualizing a body volume, wherein a data set whose data values represent said body volume is displayed two- or three-dimensionally on a display, in which method a synthesized data set is computed from at least two selected diagnostic data sets which are not identical and have a predefined spatial allocation or relationship with respect to each other, wherein each of the data values of said synthesized data set is computed as a mathematical function of at least one data value of each selected data set, and said synthesized data set is displayed on the display unit. The two- or three-dimensional visualization of said synthesized data set may comprise more various items of image information than a selected data set and may thus be more informative and more accurate in detail.

The invention also relates to a computer program product, and to a system for implementing said method.

### BACKGROUND OF THE INVENTION

1. Field of the Invention

The present invention relates to a method and a system for visualizing a body volume, in particular two- or three-dimensionally, and in particular a body volume of a human being or animal, as well as to a computer program product comprising software code portions for implementing the method in accordance with the invention.

In medical diagnostics, therapy and surgery, precise two- or three-dimensional representations of body volumes are necessary. To capture data representing the body volumes, diverse non-invasive methods of diagnosis, for example computer tomography (CT) and magnetic resonance imaging (MRI), are available. The captured data are usually digitized and subjected to image processing on a computer to visualize them. The processed image data can then be displayed two- or three-dimensionally on a monitor screen, where the image may also be rotated in three-dimensional space.

Each of the known methods of diagnosis is tailored to displaying a specific kind of tissue. For example, bone structures can be resolved particularly well by computer tomography, vascular structures can be resolved particularly well by CT angiograph methody, and hydrogenous tissue can be resolved particularly well by MRI. Thus, only a specific kind of tissue can be resolved particularly well in a two- or three-dimensional visualization of a data set captured by means of a method of diagnosis.

However, the human body consists of various kinds of tissue. Thus, for an in-depth diagnosis two- or three-dimensional visualizations captured by various methods of diagnosis must be compared with each other visually, which is laborious and results in inaccuracies in diagnosis.

2. Description of the Related Art

U.S. Pat. No. 5,335,173 discloses an image display method for medical diagnosis, in which two different data sets, captured by different methods of diagnosis and offering a particularly good resolution of a bone structure and a skin structure respectively, are displayed three-dimensionally. For improved diagnosis, a specific portion can be selected on a monitor screen displaying slice images through a three-dimensional body volume. In the selected portion of the image, the data set which represents bone structures particularly well is replaced by the data set which represents skin structures particularly well, or vice-versa. Thus, in the selected image portion, preselected image data are replaced by the corresponding data of another data set. Even exchanging image information fails to increase the accuracy and information content of the diagnosis substantially.

### SUMMARY OF THE INVENTION

It is the object of the present invention to provide a method and system for two- or three-dimensionally visualizing a body volume, wherein an even higher information content and an even higher accuracy in diagnosis is possible. This object is achieved by a method in accordance with the invention as set forth in claim 1, by a system in accordance with the invention as set forth in claim 14, and by a computer program product as set forth in claim 13. Advantageous embodiments are the subject matter of the related sub-claims.

In accordance with a first aspect of the present invention, a method for visualizing a body volume is provided, in which a data set whose data values represent the body volume is displayed two- or three-dimensionally on a display, the method comprising computing a synthesized data set from at least two selected diagnostic data sets which are not identical and which have a predefined spatial allocation or relationship with respect to each other, wherein each of the data values of the synthesized data set is computed as a mathematical function of at least one data value of each of the selected data sets, and the synthesized data set is displayed on the display.

In principle, a plurality of different mathematical functions can be used to synthesize the new data set, combining the data values of two, or more than two, data sets, preferably on a one-to-one basis, into a new data value in each case. Examples of such mathematical functions are known from the prior art, in connection with image processing or imaging. The mathematical function employed in each case can assign a data value of the synthesized data set to each data value of the at least two selected data sets. The mathematical function can alternatively also assign each of a plurality of data values of the at least two selected data sets to each single data value of the synthesized data set, such that the image data as a whole can be compressed.

Advantageously, the synthesized data set in accordance with the invention comprises image information from both the first selected data set and from the second selected data set, as well as from any other selected data set. Thus, a synthesized data set capable of combining the benefits of each of the selected data sets can be produced in accordance with the present invention by suitable image processing of one or more of the selected data sets and suitably synthesizing the image information thus processed.

In accordance with a particularly preferred embodiment of the invention, respectively different methods of diagnosis are used to capture the selected data sets. These different methods of diagnosis may be particularly well suited for resolving different tissue structures. Thus, visualizing the synthesized data set two- or three-dimensionally in accordance with the present invention may also combine the benefits of the respective methods of diagnosis used. It is of particular advantage that visualizing in accordance with the present invention comprises more varied image information and more detail accuracy, such that the accuracy of the diagnosis and also the information content of the image information can be increased.

For example, a CT (computer tomography) method may be used for capturing a first selected data set, by which method bone structures can be particularly well resolved, and an MR (magnetic resonance) method may be used for capturing the second selected data set, by which method hydrogenous tissue structures can be particularly well captured. By suitable image processing of one or more of the selected data sets and/or the synthesized data set, the data originating from the first selected data set, for example, may be particularly highlighted in the synthesized data set at the expense of the data originating from a second selected data set, as detailed in the following. The detail accuracy in visualizing the tissue structure is thus increased. Since for visualizing in accordance with the present invention, the selected data set which is based on the CT method can also be used for synthesizing the image data to be displayed, the synthesized data set can show both the bone structure and the tissue structure in particularly accurate detail and with a high information content, given suitable preparation of the data sets.

In principle, more than two selected data sets, each captured by a different method of diagnosis, may also be synthesized into a data set in accordance with the invention, said data set displaying for example more than two different tissue or bone structures. For synthesizing the synthesized data set in the aforementioned example, for instance, PET (positron emission tomographic) data can be processed in addition to the CT and MR data, and displayed collectively.

The selected data sets have a predefined spatial orientation relative to each ether, to ensure locationally accurate overlaying of the data in the synthesized data set. The selected data sets are preferably composed or processed beforehand, such that the data values of the data sets are spatially orientated in the same way. This may be achieved by composing or processing the data produced by the method of diagnosis accordingly. The spatially allocation of the respective data values of the selected data sets may, however, also be achieved by computing within the framework of synthesizing the synthesized data set. In this way, distortions of the image, such as may be due for example to the respective method of diagnosis used, can also be corrected. MR data, for example, are often distorted in the outer regions of the volume.

In accordance with a further embodiment in accordance with the invention, at least two of the selected data sets may also be computed by different image processing means from one and the same original or source data set, by means of different image processing parameters. This original data set is captured by one and the same method of diagnosis. For graphically displaying a body volume, an original data set typically needs to be graphically composed, for which image processing parameters need to be defined. Advantageously, different details in tissue structures can be highlighted particularly well by variably selecting these image processing parameters with one and the same original data set, and displayed together. Thus, in accordance with the invention even a single method of diagnosis may suffice for examination, where otherwise two or more methods of diagnosis would have been required. Further advantageously, the synthesized data set can highlight at least two different details in a tissue structure at the same time.

Expediently, the captured data sets may be captured prior to visualization and buffered on suitable data recording media. Thus, the image data can be subsequently read, for example by a data processing means, suitably composed or processed and visualized three-dimensionally, for example by an additionally consulted physician. In accordance with the present invention, however, one, more or all of the captured data sets may be captured in real time during visualization and, if necessary, additionally synthesized with buffered data sets into a new data set. Advantageously, it is possible in accordance with the invention to work in real time conditions. Information obtained during visualization, which makes changing the capture parameters of the method of diagnosis seem advantageous, for example changing the relevant capture parameters in an ultrasound diagnosis, may be applied directly and in real time in accordance with the invention, and the result displayed on the display. The accuracy of diagnosis and the image information content can thus be increased even further.

In synthesizing, it is particularly preferable to subtract and/or add the image information of two respectively selected data sets from/to each other. When employing, for example, a CT method and an MR method for capturing two selected data sets, then by subtracting the two selected data sets from each other, both the image information concerning the bone structure and the image information concerning hydrogenous tissue can be visualized together particularly well and accurately detailed. A preferred example in accordance with the invention involves the collective 2D or 3D display of brain and cranium masses. For computing the synthesized data set, however, any other method or computer algorithm known from prior art for image synthesis may in principle be used.

Optimally displaying data sets graphically, which have been captured by means of different methods of diagnosis, usually necessitates using various image display parameters.

It is thus particularly advantageous for the image to be processed and displayed by means of preset parameters, tailored to the methods of diagnosis used in each case to capture a selected data set or to highlight certain tissue structures in a selected data set. In this way, the image information of the selected data set used in each case can be displayed particularly well, without any further computing or setting steps. It is particularly preferable to use at least one parameter for image processing or imaging which influences the color and/or opacity allocation of the intensity values of the data sets. Image processing parameters are also known from the prior art which influence other graphic properties of the data sets.

For example, the preset parameter may influence a threshold value which once violated or exceeded assigns an item of brightness or color value information to an image data value. Or the parameter may influence an image gradient, such that differences between adjacent pixels can be translated into different image gradients. Furthermore, the preset parameter may also be used to influence the opacity, the color rendering used for each selected data set, or further suitable items of image information, to adapt these to the respective image display desired or to the respective underlying methods of diagnosis. It is particularly advantageous if the preset parameters used can also influence some or all of the desired items of image information.

Preferably, the aforementioned parameters used for processing or displaying the image may also be determined manually or automatically. Expediently, processing and visualizing the image is initially undertaken by means of preset parameters, and the parameters are changed as required, for example when specific details of the three-dimensional visualization need to be highlighted in particular. For this purpose the parameters may be changed manually. The operator is able to recognize the imaging result by way of the display, and to change the parameters until the image display is expedient. In this arrangement, the imaging result may be visualized three-dimensionally, whereby the three-dimensional visualization can also preferably be rotated in three-dimensional space, or displayed as a predefined two-dimensional slice image through the body volume, wherein the location of the slice through the body volume may preferably be given, e.g. by the operator. In this way, the operator is able to directly affect visualization and optimize the parameters, in order to achieve optimal detail accuracy in visualization and optimal image information.

In accordance with the invention, however, the parameters may also be automatically optimized, by means of an optimization method, various kinds of which are known from the prior art. Preferably, the operator is thereby able to define the image information for which display is to be optimized, for example the bone structure or the vascular structure in the body volume, or a specific slice image or body part volume.

It is particularly preferable to display the data in the synthesized data set, these stemming from various selected data sets, i.e. being captured by means of various methods of diagnosis or derived from one and the same original data set by the use of various image processing parameters, in various colors. Advantageously, the various structures, for example bones, vascular or tissue structures, permit direct recognition. This has proven particularly advantageous in border or transition areas between differing tissue structures.

In visualization, the brightness of the various color values used, as assigned to the selected data sets, can be varied with time, for example periodically, continuously or periodically cycled so that contrasts between different structures can be perceived directly in sequence from various differences in brightness.

Three-dimensional visualizations of each of the seleted data sets and/or two-dimensional slices through the body volumes are preferably displayed on the display in addition to the two- or three-dimensional visualizing of the synthesized data set, in particular axially, sagittally or coronally. Advantageously, both the synthesized image information in which individual tissues are highlighted in particular and the data set selected in each case may be displayed within a restricted space; in a first segment of the display, for example, data stemming from a CT image, and in another segment, data stemming from an MRI method, and in another segment, data stemming from a PET method, in another segment the synthesized data, etc.

Each visualization displayed on the display can preferably be freely rotated in three-dimensional space, individually and independently of any other visualization, for example by operating a trackball or other operational control. It is particularly preferred if parts of the body volume can also be displayed in definable magnification and three-dimensional orientation.

The data sets employed can in principle be captured by any method of medical diagnosis suitable for the three-dimensional display of body volumes. Particularly preferred for use in capturing data sets are the following methods: CT, CT-A, MRI, MR-A (magnetic resonance angiograph methody), functional MRI or fMRI, PET (positron emission tomography), MEG (magnet encephalography), SPECT and ultrasound. However, the invention is not restricted to the aforementioned methods.

In accordance with a further aspect, the present invention comprises a computer program product, directly loadable into the RAM of a digital computer and comprising software code portions for implementing the aforementioned steps in the method when the product is run on a computer. The computer program product may be stored on any data recording media, for example magnetic or magoptical disks, tapes, etc., or can be loaded via a network or the Internet. In particular preference, the computer program product can also be used by several computers at the same time.

In accordance with a further aspect, the present invention comprises a system for two- or three-dimensional visualization of a body volume, including a data processing means for computing a synthesized data set from at least two selected diagnostic data sets which are not identical and have a predetermined spatial allocation or relationship with respect to each other, such that the data values of the synthesized data set are each computed as a mathematical function of at least one data value of each of the selected data sets, and also including a display for displaying the synthesized data set whose data values represent the body volume two- or three-dimensionally.

A means may be provided for inputting the selected data sets into the data processing means. The input means may be a typical data interface with external data storage means, for loading buffered data sets into the system, or at least one input means may be coupled to a medical diagnosis apparatus, to capture a data set such that the system in accordance with the invention can then also be operated in real time.

The at least two selected data sets may be selected by means of a menu control, for example manually by means of a computer program selecting the data sets on the basis of defined parameters, in particular automatically, or in some other way.

The system is preferably designed as a commercially available work station, the aforementioned means preferably being realized in the form of software. The aforementioned steps in the method are also preferably realized in the form of software, or software modules or software code portions.

The synthesized data sets and/or the selected data sets and/or slice images obtained from the selected data sets are preferably displayed at predetermined points on a display, such that the operator has extensive image information and options for diagnosis at his disposal, in a compact form.

The system in accordance with the invention may also be realized as a module in a typical system for capturing data sets with the aid of an imaging method of diagnosis, for example in a computer tomograph, whereby the other selected data set or sets can then be transferred from a data storage or a network.

### BRIEF DESCRIPTION OF THE DRAWINGS.

The invention will now be described by way of example and with reference to the attached drawings, in which:

FIG. 1 is a schematic diagram explaining the method and system in accordance with the invention;

FIG. 2 is an example of a display visualizing synthesized data and selected data sets three-dimensionally and side-by-side;

FIGS. 3a,3b show, in two different parameter settings, a window for setting parameters influencing the image display of a selected data set;

FIGS. 4a,4b show, in two different parameter settings, another window for setting parameters in the image display of another selected data set;

FIG. 5 is a three-dimensional visualization of a synthesized data set, as well as an enlarged view of a portion thereof.

Identical reference numerals in the drawings denote identical or equally acting elements or steps in the method.

### DESCRIPTION OF THE PREFERRED EMBODIMENTS.

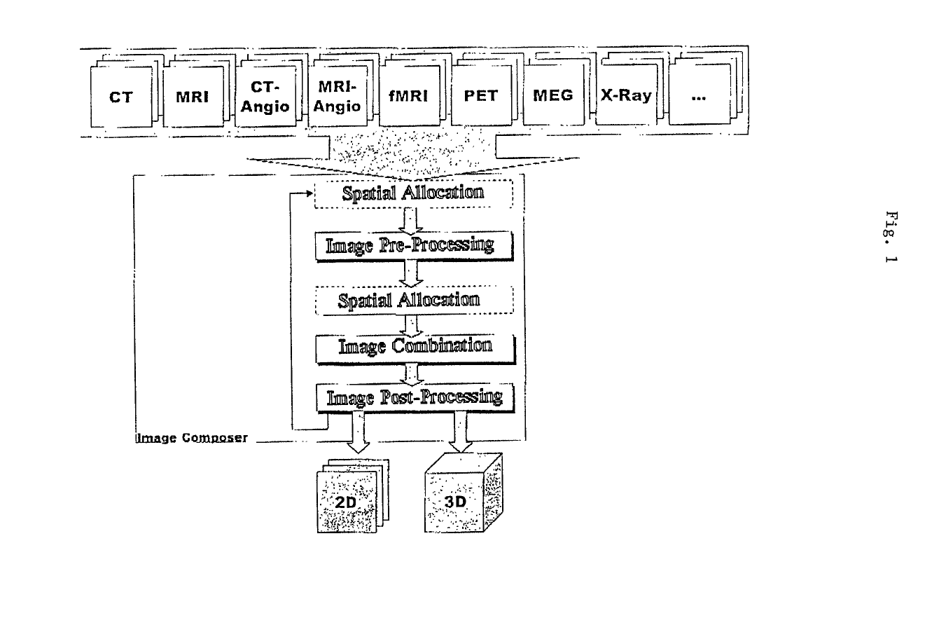

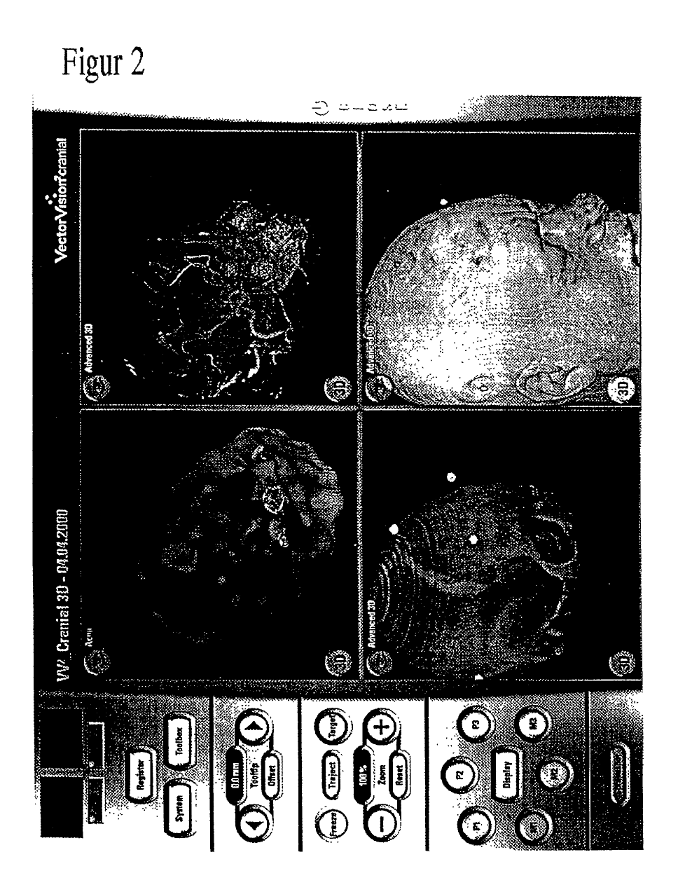

FIG. 1 shows a schematic flow diagram explaining the method and system in accordance with the invention. The system 1 comprises an image composer 2, a display unit 6 for displaying two-dimensional slice images or sectional views, as well as a display unit 7 displaying data sets three-dimensionally. The display units 6 and 7 may form a common display unit.

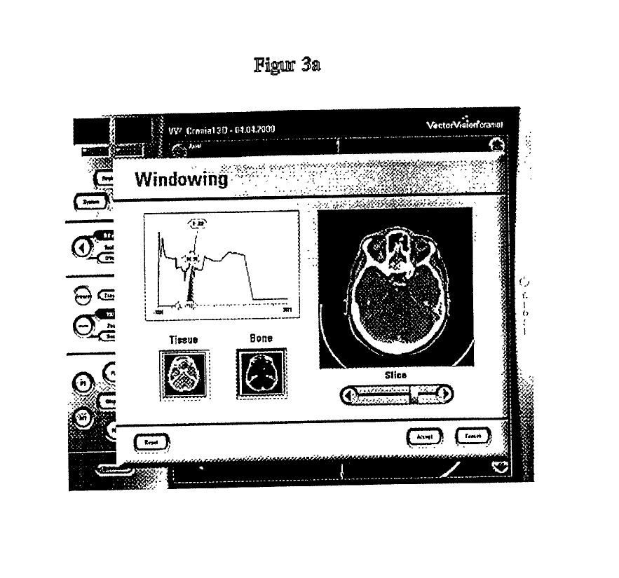

A number of different diagnostic data sets, captured by various methods of diagnosis, may be inputted into the image composer 2. As shown in FIG. 2, data sets may be captured using a CT method (computer tomography), a CT angiograph method, a magnetic resonance method (MR), an MR angiograph method, a positron emission tomography method (PET), a functional MRI method (fMRI), an x-ray rotational angiograph method, a 3D ultrasound method, MEG (magnetic encephalography), or any other imaging method of medical diagnosis. The different data sets 8 inputted into the image composer 2 may, however, also be derived from one and the same data set by differing methods of image preprocessing, especially for variously highlighting differing tissue structures by means of differing image parameters, each being used for a different selected data set 8.

As indicated in FIG. 1 by the layers of the data sets 8, the input data sets 8 are typically organized in two-dimensional layers, wherein the sum of the 2D layers of each data set represents the body volume to be displayed. For two-dimensional display, axial, sagittal or coronal slices through the body volume are particularly suitable, although input data sets may also be organized differently.

Each data set can be stored in a data storage means (not shown) and retrieved by the image composer 2, for example as selected by the operator. For this purpose, the composer 2 is connected to the data storage means via an interface, a network or a comparable means. At least one of the data sets may, however, also be captured in real time by a diagnostic device. The image composer 2 comprises a section for spatial allocation R, R′, an image combination section 3 and at least one imaging section 5, 5′. Each of the sections is preferably implemented as software. Once selected by an operator or by a computer program running on the image composer 2, the image combination section 3 combines or synthesizes at least two of the data sets 8 in accordance with a definable image combination algorithm. This algorithm realizes a mathematical function which preferably assigns each new data value to the data values of the selected data sets 8 with a corresponding spatial location, on a one-to-one basis. The sum of the data values computed in this way forms the synthesized data set. The mathematical function may also combine a number of respective data values of the selected data sets into a single data value of the synthesized data set with a corresponding spatial allocation or relationship. In the simplest case, adding and/or subtracting data values to/from each other of two selected data sets 8 may be employed as the image combination algorithm, or also other image combination algorithms suitable for diagnostic visualization.

In order that the selected data sets may be superimposed with exact positioning, the spatial geometry of the selected data set, and also other parameters, such as for example the zoom factor of each data set, is taken into account, so that the data sets can be captured in various reference systems. Preferably, the selected data sets are spatialy arranged precisely with respect to each other. The spatial allocation or relationship R, R′ may be rigid, i.e. non-variable. As indicated by the broken line frames, the spatial allocation R, R′ may also be elastic, i.e. variable, so that for example distortions occurring in a selected data set 8 relative to a second selected data set 8 can be corrected prior to or during synthesizing. The spatial allocation R of the data values may be achieved prior to image pre-processing 5 or thereafter (R′).

In section 3, the selected data sets 8 are combined with each other by synthesizing the image information or image information derived therefrom, by suitable mathematical functions.

In the image composer 2, at least one of the selected data sets can be subjected to 2D or 3D imaging or image processing, in order for example to highlight tissue structures in the data set particularly well. For medical diagnostic visualization methods, suitable image processing methods are known. Parameters are required for each of the image processing methods employed. These image processing parameters can be predefined, or defined manually or automatically, as explained below.

Once synthesized, the synthesized data set is displayed in a two-dimensional slice display on the display unit 6, wherein location and orientation of the slice through the body volume may be predefined, for example by a slider, a trackball or plus/minus buttons on a touch screen.

A three-dimensional visualization is also computed from the computed, synthesized data set, and displayed on the display unit 7. This visualization can be rotated in any way in three-dimensional space, for example by menu control, trackball or plus/minus buttons on a touch screen, wherein portions of the body volume may be displayed enlarged or rotated.

As shown schematically in FIG. 1, the display shown on the display unit 6 or 7 comprises image information from each of the selected data sets 8. For example, the image composer 2 may select a CT image and an MR image. The CT image provides a particularly good resolution of the bone structure, in the present case of a skull. The magnetic resonance image (MR) provides good resolution of the brain structure, and where necessary of the vascular structure too, but not of the bone structure. The synthesized data set thus simultaneously comprises image information relating to the bone structure, the vascular structure and the brain structure. If a PET image is additionally selected, with which metabolically active areas in particular may be visualized, these areas may also be displayed in the synthesized data set. For synthesizing the data set, the selected data sets may be added, for example with predefined weighting or opacity and/or color rendering of the selected data sets.

For synthesizing the data set, each of two selected data sets may also be subtracted from one another. When, for example, a data set captured by means of an MR method is subtracted from a data set captured by an MR angiograph method, brain structures can be practically eliminated from the image, excepting the vascular structure. This may necessitate a suitable weighting of the respective selected data sets, or a suitable image processing of the selected data sets, as detained below.

A mixed data set may also be displayed on the display unit 7, said data set representing a three-dimensional partial slice through a synthesized data set, for example the bone or skin structure of a human cranium, wherein the upper part of the cranium is displayed cut away and this partial slice and the synthesized data set are superimposed, for example the three-dimensional vascular structure in the human cranium, projecting three-dimensionally through the human cranium, out from the slice plane.

To enhance the information content of the synthesized data set, at least one of the selected data sets 8 may be subjected to image processing 5, 5′ to effectively highlight those structures contained in the selected data set which can be captured particularly well by the method used for capturing the selected data set. For this purpose, preset parameters may be used which are known to be typically suitable for displaying data sets captured with the aid of the methods of diagnosis employed. However, the parameters may also be determined manually or automatically.

For methods of medical visualization, various parameters particularly suited to highlighting details in the display of images are known. These are typically parameters influencing the color and opacity assignment of the intensity values of the data sets. A few examples of preferred parameters are cited in the following. A threshold value, for example, may be set by the parameter, such that pixels whose value exceeds the threshold value are displayed bright and/or colored, and pixels whose data value does not reach the threshold value are displayed with a constant color or brightness, for example in black alone. A color and/or brightness gradient may also be influenced by the parameter, in order to scale the data values. The opacity or transparency of the image data values of a selected data set may also be influenced by the parameter, such that in a first data set displayed semi-transparent, three-dimensionally representing a brain structure for example, a second set is recognizable, representing for example the vascular structure in the brain structure. The parameter may also influence the color used to display a synthesized data set or a selected data set. Further image processing parameters are known from the prior art.

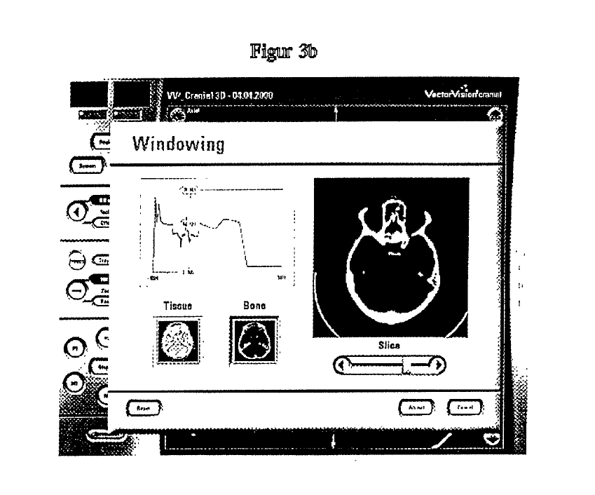

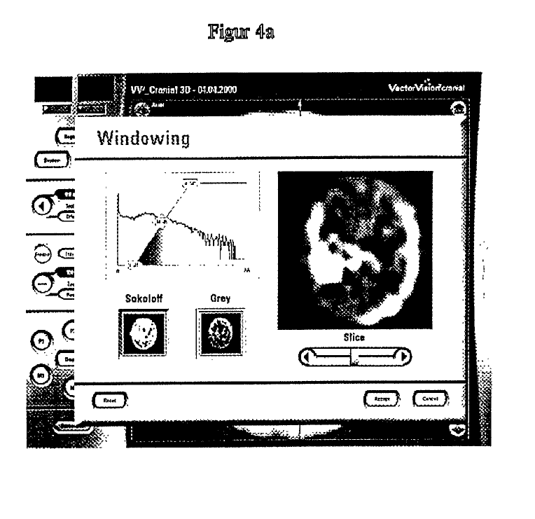

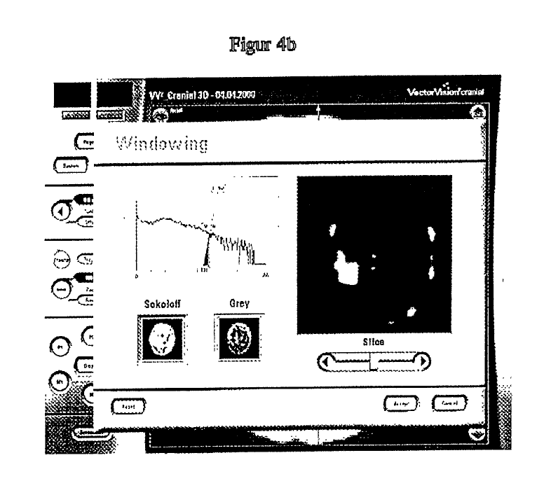

To define the image processing parameter manually, a slice image is displayed by a selected data set on the display unit 6 as shown in FIG. 1, wherein the three-dimensional location and orientation of the slice image may be predefined. By means of a parameter setting device, schematically indicated by the reference symbol I, one or more image processing parameters are modified until the slice image shown on the display unit 6 or the three-dimensional display on the display unit 7 exhibits the desired resolution and image information. This will now be explained with reference to FIGS. 3 and 4. To establish the image processing parameters, the loop L as shown in FIG. 1 can be run through a number of times. Once the image processing parameters have been defined manually, the body volume is visualized three-dimensionally on the display unit 7 as shown in FIG. 1, by using the defined image processing parameters. Alternatively, the image processing parameters may also be defined directly by way of the three-dimensional visualization on the display unit 7 which, however, necessitates as a rule a greater compution time.

The synthesized data sets thus obtained may be stored separately or together with the selected data sets and/or supplementarily with all of the captured data sets.

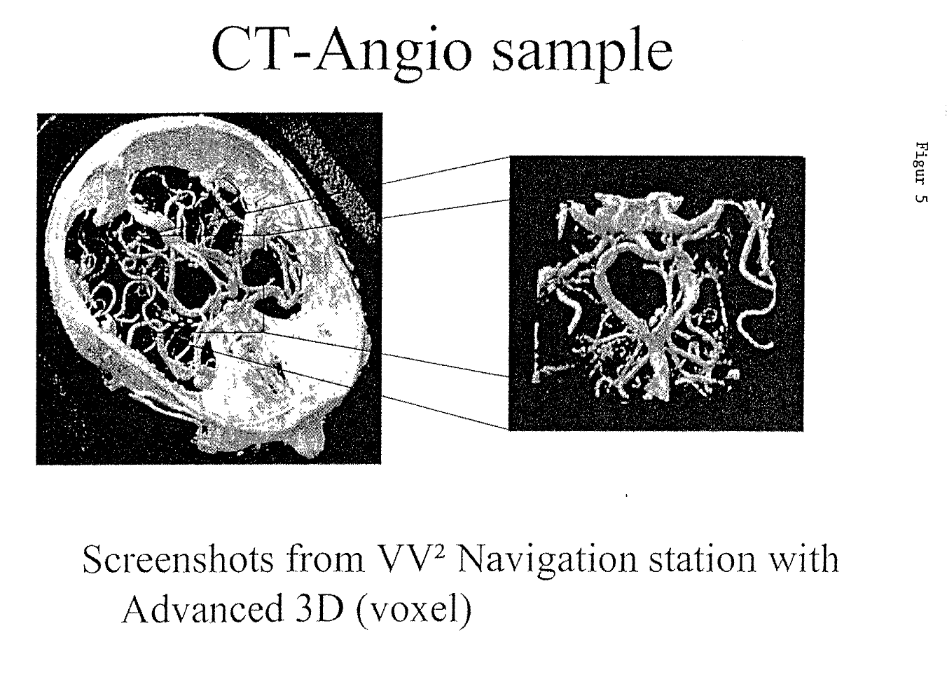

FIG. 2 illustrates a preferred example of a 3D display on the display unit 7. The 3D display 10 as shown in FIG. 2 comprises four image segments 12 in the right-hand portion of the image, in each of which three-dimensional visualizations are displayed which can be spatially rotated or enlarged, together or independently of each other. As shown in FIG. 2, PET data are displayed three-dimensionally in the left-hand upper segment of the image, image data captured by MR angiograph methody in the right-hand upper segment, CT image data in the left-hand lower segment, and MR image data in the right-hand lower segment. The display unit 7 additionally comprises a segment for three-dimensional visualization of a synthesized data set as shown in FIG. 5.

A control panel 11 is arranged in the left-hand portion of the window, as shown in FIG. 2, and includes a number of control elements, for example, sliders or buttons on a touch screen, for setting the processing and manipulating of the image, and recording of the data.

FIGS. 3 and 4 illustrate the 2D display unit 6 and 6′ respectively, including a window and an operator surface for defining image processing parameters, specifically two different image processing parameters in each case. As shown in FIGS. 3 and 4, the window 6, 6′ comprises a display 15, 15′ for displaying a two-dimensional slice image of the selected data set through the body volume in each case. With the aid of the slider 16, the spatial location of the slice image in the body volume can be changed. Two buttons 14, 14′ are provided in the lower part of the window, for defining the standard settings for the image processing parameter or parameters. A graphic display 13, 13′ for visualizing the currently set image processing parameter, and the tools indicated by shiftable rectangles for changing each image processing parameter, are displayed in the left-hand upper part of the window. In the toolbox window 13, the x-axis corresponds to the threshold value and the y-axis to the frequency of the image data values having a specific image density, wherein the image density, for example for CT data, is displayed in Houncefield units.

FIG. 3a illustrates a slice image through a CT image, the image processing parameters having been selected so that both bone structures and tissue structures of the skull are recognizable. As can be seen by comparing the displays 15 as shown in FIGS. 3a and 3b, the image gradient, which is converted into brightness levels of the image, is selected less steep for a comparable threshold value in FIG. 3a than in FIG. 3b. Whilst, as shown in FIG. 3a, both bone and tissue structures are recognizable, only bone structures are in practice still recognizable in FIG. 3b. By shifting the respective tools in the graphic display 13, the image processing parameters can be varied until the image shows the desired resolution. To optimize the image processing parameters, the location of the slice image in the body volume may also be varied by shifting the slider 16.

By pressing the buttons “tissue” or “bone”, preset image processing parameters can be activated, with which tissue structures or bone structures known from experience may be particularly well highlighting.

As shown in FIGS. 4a and 4b, a slice image through a PET data set comprising two different image processing parameters is displayed in the window 6′. By shifting the tools in the setting window 13′, the desired tissue structures, having enhanced metabolic activity, can be highlighted in the display area 15′.

The image processing parameters used to visualize the synthesized data set may also be defined or optimized automatically. In this way, a slice image can be defined on the display unit 6, which displays the image information to be highlighted, for example a bone structure, particularly well. Algorithms are known from prior art for defining the relevant image processing parameters. Optimization may also be achieved in a 3D visualization.

The parameters used for image processing and visualization can be changed at any time, for example during an operation, to adapt to the different steps in surgery.

As shown in FIG. 5, the synthesized data set is visualized three-dimensionally in a segment 12 of the display unit 7, by use of the preset or defined image processing parameters. The display can be rotated and enlarged (window 20) at will in three-dimensional space.

Instead of visualizing different tissue structures three-dimensionally on display unit 7, or two-dimensionally on display unit 6, constant over time, the color or brightness assigned to the various tissue structures can be changed over time, in accordance with a preferred embodiment, such that two different tissue structures can for example be visualized with the same intensity at a first point in time, whereas at a second point in time the two tissue structures are displayed with differing intensities, and at a third point in time one of the tissue structures may be visualized with disappearing intensity, etc., such that the observer can alternately concentrate on different tissue structures. The brightness or intensity on the display unit 6 or 7 can be constantly or incrementally varied, as instructed by the operator.

For implementing the method as described above, a computer program product is also disclosed, comprising software code portions for implementing the aforementioned steps in the method when the software code portions are loaded into the RAM of a digital computer.

The present invention is not restricted to the methods of diagnosis cited above for capturing image data sets. In accordance with the present invention, any method of three-dimensional diagnostic visualization may be used, wherein each of the image data sets may be composed and processed in any way, for synthesizing the synthesized data set.

In the foregoing description preferred embodiments of the invention have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

What is claimed is:

1. A method for visualizing a body volume, wherein a data set whose data values represent said body volume is displayed two- or three-dimensionally, in which method a synthesized data set is computed from at least two selected diagnostic data sets which are not identical and have a predefined spatial allocation with respect to each other, wherein each of the data values of said synthesized data set is computed as a mathematical function of at least one data value of each selected data set, and said synthesized data set is displayed on the display unit.

2. The method as set forth in claim 1, wherein at least two said selected data sets are selected from data sets, each of which is captured by a different method of diagnosis.

3. The method as set forth in claim 2, wherein at least two of said selected data sets are computed by different image processing means, using different image processing parameters, from one and the same original data set captured by a method of diagnosis.

4. The method as set forth in claim 1, wherein said selected data sets are inputted to a data processing means.

5. The method as set forth in claim 1, wherein image processing is carried out, to synthesize said synthesized data set.

6. The method as set forth in claim 5, wherein said synthesized data set is derived by subtracting and/or adding two selected data sets, respectively.

7. The method as set forth in claim 5, wherein image processing is carried out by means of at least one parameter influencing the color and/or opacity allocation of the intensity values of the data sets.

8. The method as set forth in the preceding claim, wherein the result of said image processing is visualized as a predefined two-dimensional slice image through said body volume.

9. The method as set forth in claim 1, wherein in said synthesized data set different color values are assigned to data from different selected data sets.

10. The method as set forth in the preceding claim, wherein the display parameters of at least one selected data set in said synthesized data set are varied over time.

11. The method as set forth in claim 1, wherein in addition to said two- or three-dimensional visualization of said synthesized data set, three-dimensional visualizations of each of said selected data sets and/or two-dimensional slices through said body volume are displayed on said display unit.

12. The method as set forth in claim 1, wherein said data sets are captured by means of CT, CT-A, MRI, MR-A, fMRI, PET, MEG, SPECT or ultrasound.

13. A computer program product, directly loadable into the RAM of a digital computer, and comprising software code portions for implementing the steps in the method as set forth in claim 1, when said product is run on said computer.

14. A system for visualizing a body volume, comprising:
a) a data processing means for computing a synthesized data set from at least two selected diagnostic data sets which are not identical and have a predefined spatial allocation with respect to each other, such that each of the data values of said synthesized data set is computed as a mathematical function of at least one data value of each of said selected data sets, and
b) a display for displaying said synthesized data set, whose data values represent said body volume, two- or three-dimensionally.

15. The system as set forth in claim 14, including an input means for inputting said selected data sets into the data processing means.

16. The system as set forth in claim 14, including an image processing means for subjecting at least one of said selected data sets and/or said synthesized data set to image processing.

17. The system as set forth in the preceding claim, wherein said image processing means is devised to carry out image processing by means of at least one parameter influencing said color and/or opacity allocation of said intensity values of said data sets.

18. The system as set forth in claim 14, further comprising adding and/or subtracting means, for adding and/or subtracting each of said two selected data sets.

19. The system as set forth in claim 14, further comprising a display, for displaying a two-dimensional slice image through said body volume for a selected data set or said synthesized data set.

20. The system as set forth in claim 14, further comprising a display, for displaying a three-dimensional visualization of said synthesized data set and three-dimensional visualizations of at least one image selected from a group consisting of said selected data sets and of two-dimensional slice images of said synthesized data set and of two-dimensional slice images of said selected data sets through said body volume.

21. The system as set forth in claim 14, wherein said input means is connected to a data storage means.

22. The system as set forth in claim 14, wherein said input means is connected to a means for caputuring data sets by means of CT, CT-A, MRI, MR-A, fMRI, PET, MEG, SPECT or ultrasound.
